(19) United States
(12) Patent Application Publication
(10) Pub. No.: US 2025/0281597 A1
(43) Pub. Date: Sep. 11, 2025

(54) A VACCINE FOR THE PROTECTION OF PIGLETS AGAINST SWINE INFLUENZA A VIRUS INFECTION

(71) Applicant: Intervet Inc., Rahway, NJ (US)

(72) Inventors: Basav Hangalapura Nagaraj, Wageninger (NL); Mark A. Mogler, Ames, IA (US); Ruud Philip Antoon Maria Segers, Boxmeer (NL)

(73) Assignee: Intervet Inc., Rahway, NJ (US)

(21) Appl. No.: 18/702,577

(22) PCT Filed: Oct. 24, 2022
(86) PCT No.: PCT/EP2022/079532

§ 371 Date: Apr. 18, 2024

(30) Foreign Application Priority Data
Oct. 25, 2021 (EP) 21204364.0

(51) Int. Cl. A61K 39/145; A61K 39/00; A61K 39/08; A61K 39/108; A61P 37/04

(52) U.S. Cl. CPC A61K 39/145; A61K 39/0258; A61K 39/08; A61P 37/04; A61K 2039/5256; A61K 2039/552

(57) ABSTRACT

The present invention pertains to the use of a vaccine based on an alphavirus RNA replicon particle (αRP) vector encoding an antigen of an IAV-S for the passive vaccination of piglets against a pathogenic infection with swine influenza virus.

### GENERAL FIELD OF THE INVENTION

The invention concerns a swine influenza A viruses (IAV-S) vaccine that can be used for the passive immunization of progeny piglets by active immunization of female pigs.

### BACKGROUND OF THE INVENTION

Influenza A viruses (IAV) create a significant burden on human and animal health, worldwide. IAV is categorized into different subtypes based on its viral surface glycoproteins, hemagglutinin (HA) and neuraminidase (NA). IAV infects poultry, pigs, horses, cats, dogs, marine mammals (e.g., whales), bats and humans. Wild waterfowl and shorebirds (ducks, geese, swans and gulls) are the natural reservoirs and they can be infected with 16 different HA and 9 different NA subtypes (Webster et al., Microbiol Rev 56:152-179, 1992).

Influenza A virus in swine (IAV-S) is a serious respiratory pathogen of domestic pigs that has proven to be economically costly, particularly to the livestock industry, worldwide (Holtkamp et al., The American Association of Swine Veterinarians Annual Meeting, 2007). It is characterized by a sudden onset of respiratory illness, and is usually accompanied by anorexia, lethargy, and fever. In addition to the clinical complications associated with IAV-S in production animals, there have been published reports implicating swine in the transfer of influenza viruses to humans (Myers et al., Clin Infect Dis, 2007; 44 (8), 1084-8, Krueger and Gray, Curr Top Microbiol Immunol 370:201-225, 2013), which represents a significant public health threat providing an even greater incentive to control IAV-S in swine herds.

In response to this problem, many swine farmers now vaccinate their pigs against IAV-S employing commercially available vaccines. However, controlling IAV-S with the conventional vaccines is difficult because many diverse IAV-S strains co-circulate in the field and continue to evolve (Gao et al., J Gen Virol 98 (8), 2001-2010, 2017). The diversity and mutability of IAV-S are caused by the virus's genetic structure. Like other influenza A viruses, IAV-S has genes encoded on eight segments of RNA and a genome replication machinery that introduces frequent mutations. These genetic characteristics enable IAV-S to make rapid adaptions, including escape from existing neutralizing antibodies induced by exposure to previous strains.

Classification of influenza A viruses starts with subtyping of HA and NA, the two major glycoproteins on the virus surface. HA protein mediates attachment and fusion of the virus to host cells. Neuraminidase is an enzyme that functions in the final stage of the influenza virus replication cycle by cleaving newly formed viral particles from the host cell, thereby enabling the new progeny virus to spread and infect other cells.

Whereas human influenza A usually has 1 or 2 dominant strains circulating globally during a given influenza season, many more strains of IAV-S co-circulate simultaneously, with these strains differ between geographic regions. Similarly, IAV-S strains are also antigenically variable, but mainly contain an H1 or H3 subtype of HA, and a N1 or N2 subtype of NA. Within each HA and NA subtype of IAV-S there is further phylogenetic diversity.

In the USA swine population there are four predominant phylogenetic clusters of H1 (gamma, delta1, delta2, pandemic), two predominant clusters of H3 (cluster IV and human-like), two predominant clusters of N1 (classic, pandemic), and two predominant clusters of N2 (N2-1998 and N2-2002). (See, Anderson et al., Influenza and other Respiratory Viruses 7 (Suppl. 4), 42-51, 2013; and Anderson et al., mSphere 1 (6) e00275-16:1-14 (2016)).

In Europe there are three major lineages of H1 (Eurasian avian like, Scotland/410440/1994-like H1 and pandemic), one major lineage of H3 (Gent/1/1984-like H3), two major lineages of N1, two major lineages of N2 and two minor lineages of N2 (Watson et al., J. Virol., 89:9920-9931 (2015); doi: 10.1128/JVI.00840-15).

As a consequence of the continuous emerging of variant IAV-S strains, commercially available whole virus inactivated vaccines often do not protect against new and emerging virus subtypes/clusters, and offer only limited protection against heterosubtypic challenge, since the antigens do not match all contemporary strains circulating in the field (Lee et al., Can J Vet Res 71 (3), 207-12, 2007; Vincent et al., Vaccine 28 (15), 2782-2787, 2010). Thus, such vaccines must be periodically updated to match currently circulating strains.

Common strategies to prevent piglets from infectious diseases include the vaccination of pregnant female pigs pre-farrowing and/or piglet progeny, with whole virus inactivated vaccines. Vaccinated pregnant female pigs transfer their protection to piglets in colostrum and subsequent milk (passive immunity). In practice, the passive immunity of a piglet may be obtained from its female parent or from a different female pig vaccinated pre-farrowing. The colostrum contains a battery of immune system components to help the piglet survive until it develops its own active immunity. The level of antibodies in the colostrum is 60 times higher than in the milk. Around 65-90% of these antibodies consist of the IgG type, which provides systemic protection. The absorption of these IgG immunoglobulins by piglets is not selective; they are present in the plasma 2 hours after colostrum ingestion and peak at 12 hours. The antibody profile of colostrum and the passive protection induced in piglets is dependent on the antigens that the mother is exposed to and the duration between the antigen exposure and the farrow. During lactation, IgG immunoglobulins are progressively replaced by IgA, which functions to protect the intestinal mucosa of the piglet. The gut closes to the absorption of antibodies and cells after approximately 36 hours, piglets continue to receive passive mucosal IgA antibodies in their gastrointestinal tract from the milk.

In the art, in addition to whole virus inactivated vaccines, several other types of vaccines for use in pigs are described (reviewed by Opriessnig et al. Porcine Health Management, 2021, 7, 1). Broadly, these can be categorized into whole pathogen attenuated-live vaccines, chimeric (attenuated-live or inactivated) vaccines, subunit vaccines, live mammalian virus vectors (replicating or replication deficient) vaccines and nucleic acid-based vaccines (DNA plasmids or mRNAs).

WO 2019/121513 and WO 2019/110481 describe the use of replication deficient alphavirus RNA replicon particle vectors for the active vaccination of piglets against IAV-S.

It is an object of the invention to develop novel strategies and vaccines for pigs that are safe, effective against virulent challenge by IAV-S and can be rapidly altered to antigenically match emerging strains.

### SUMMARY OF THE INVENTION

In order to meet the object of the invention a vaccine is provided comprising an immunogen and a pharmaceutically acceptable carrier for use in a method to protect a piglet against a pathogenic infection with swine influenza A virus (IAV-S) by passive immunization of the piglet through ingestion of colostrum or milk from a female pig actively immunized with the vaccine, wherein the immunogen is an alphavirus RNA replicon particle (αRP) vector encoding an antigen of an IAV-S

Up to weaning, the protection of piglets against local pathogens, such as porcine epidemic diarrhea virus (PEDV), E. coli, Clostridium, parvovirus, PCV2, Rota virus, TGE virus, is mainly due to immunity conferred by milk (lactogenic immunity). This immunity is dependent on the immune activation at induction sites of a sow and on the transfer of activated B cells into mammary glands with local production of secretory IgA. In sows, immune activation occurs in the GALT (Gut-associated lymphoid tissue) with lymphocyte recirculation between intestine and mammary gland and in the BALT (Bronchus-associated lymphoid tissue) with B cell recirculation between respiratory system and mammary gland.

For optimal lactogenic immunity in the local mucosa of pigs, in addition to systemic immunity, the immunising antigen in a vaccine must also stimulate a local immunity mainly by acting on the lymphatic tissues of the intestines (“Peyer's patches”). This results in the production of IgA just below the surface layer of mucosal tissues, such as in the gut and respiratory tract. The IgA passes through the cells of the mucous membrane and attaches to another molecule called the secretory component. It is then called secretory IgA and consists of two IgA antibody molecules joined together by the secretory component. This combination increases their potency, makes them resistant to digestion by gut enzymes, and more readily absorbed by mucus. Since the mucus coats the whole lining of the intestines and the respiratory tract, the secretory IgA acts as a shield against potentially pathogenic infections.

The current invention results from an unexpected finding that, despite the fact that experiments show that a vaccine based on an αRP vector failed to afford passive protection in piglets against a pathogenic infection with a swine pathogen that, like IAV-S, also replicates locally in mucosal tissue, PEDV (Example 3), in contrast, the active vaccination of sows with an αRP IAV-S vector based vaccine did provide passive protection in the sows' piglets (Example 2).

### LEGENDS TO THE FIGURES

FIG. 1. IAV-S haemagglutination inhibition (HI) titers gilt colostrum, sow sera and piglet sera. Eurasian-avian (France/53-130065) lineage IAV-S strain specific HI titers (log 2) measured in sow colostrum (n=3) collected at farrow, sow sera (n=3) collected at farrow and piglet sera (n=18-27) collected at 1 week of age.

FIG. 2. Weighted lung lesion scores with mean+SD of the piglets measured at 3 days post challenge. Average values+SD were based on 14 (IAV-S RP+ColiClos IM), 16 (IAV-S RP XSolve50 IM), 16 (IAV-S RP XSolve50 ID) and 15 animals (PBS+ColiClos IM) belonging to three litters each. Three gilts per group were vaccinated at 6 and 2 weeks pre-farrow and the off-springs were infected at 4 WOA and necropsied 3 days post infection to evaluate lung lesion scores.

FIG. 3. IAV-S titers with mean+SD of the piglet lung tissues collected at 3 days post challenge. Average values+SD were based on 14 (IAV-S RP+ColiClos IM), 16 (IAV-S RP XSolve50 IM), 16 (IAV-S RP XSolve50 ID) and 15 animals (PBS+ColiClos IM) belonging to three litters each. Three gilts per group were vaccinated at 6 and 2 weeks pre-farrow and the off-springs were infected at 4 WOA and necropsied 3 days post infection to measure viral load in the lungs.

FIG. 4. PEDV-neutralizing antibodies titers in the sow sera that were vaccinated with RP-PEDV or placebo. The PEDV neutralizing titers or serum neutralizing titers were determined as FFN titer using FFN assay against cell culture adopted PEDV strain USA/Colorado/2013. DPV=Day post vaccination, DPF=Days post farrow. Values below dotted line are considered to have no PEDV neutralizing activity.

FIG. 5. PEDV-neutralizing antibodies titers in the sera samples of pigs born to sows that were vaccinated with RP-PEDV or placebo. The PEDV neutralizing titers or serum neutralizing titers were determined as FFN titer in the pig sera collected at the day of challenge (3-5 days of age) using FFN assay against cell culture adopted PEDV strain USA/Colorado/2013.

FIG. 6. The percentage mortality in pigs due to PEDV challenge. Three to five day old pigs that were born to sows that were vaccinated with RP-PEDV or placebo were intragastrical inoculated with 10⁶ TCID50 of PEDV/Colorado/2013 challenge strain in a total volume of 3 mL per pig. The mortality of infected pigs per litter were recorded until 14 days post challenge and the percentage mortality of pigs per litter is presented in the figure.

### DEFINITIONS

A vaccine is a composition suitable for administration to an animal, comprising one or more antigens of an infectious agent in an immunologically effective amount, typically combined with a pharmaceutically acceptable carrier, which upon administration to the animal induces an immune response that protects the animal against a pathogenic infection with the infectious agent.

Protection or protect against a pathogenic infection with an infectious agent means arriving at protective immunity in an animal, i.e. aiding in preventing, ameliorating or curing (an) adverse effect(s) caused by the infection with that agent, for example, by reducing the number or the duration of the viral replication in the animal, by shortening or reducing the number, the intensity, or the severity of tissue lesions, or by preventing one or more clinical signs.

A pig or swine refers to any animal of the family of Suidae, in particular to animals of the genus Sus, for example: a wild- or a domestic pig, wild boar, babirusa, or warthog.

A piglet is a young progeny of a female pig.

A sow is an adult female pig that has already farrowed a litter of piglets.

A gilt is a young female pig that has not yet produced a litter.

Colostrum is the first milk produced by a sow or gilt with each litter of piglets.

Active immunization as used herein is the stimulation of a pig's immune system following the exposure of the pig's body to a foreign antigen resulting in the generation of antibodies and immune cells. The antigen can be in the form of an infectious agent, an inactivated form of the agent or an immunogenic component of the agent.

Passive immunization as used herein concerns the transfer of a female pig's immunity (antibodies and/or immune cells) to newborn piglet progeny through ingestion of colostrum and, preferably also subsequent milk. In practice, passive immunity of a piglet progeny may be obtained from its female parent or from a different female pig vaccinated pre-farrowing.

An alphavirus is a genus of RNA viruses, belonging to the Togaviridae family, that are small, spherical, enveloped, positive-sense ssRNA viruses (Fields Virology: Emerging Viruses, authors: Howley, Knipe and Whelan, ISBN/ISSN 9781975112547, 2020).

As used herein, the term alphavirus RNA replicon particle (αRP), is an alphavirus-like-particle comprising a modified RNA viral genome that lacks one or more coding sequences for structural proteins, that if they were present, would enable the successful propagation of the parental virus in cell cultures or animal hosts, packaged in viral structural proteins, e.g., the capsid and glycoproteins, which also are derived from an alphavirus, for example, as described by Pushko et al., (Virology 239, 389-401, 1997). Consequently, αRPs are able to enter an animal's host cell and perform one round of viral genome amplification without the ability to form new particles. The replicon particle does not propagate from the infected cell, as it lacks the necessary structural protein-coding sequence(s) particles (see also: Vander Veen et al., 2012, Anim. Health. Res. Rev., vol. 13, p. 1-9; and: Kamrud et al., 2010, J. Gen. Virol. 91, 1723-1727).

Several alphavirus species have been used to develop αRP based vaccines, e.g.: Venezuelan equine encephalitis virus (VEEV) (Pushko et al., 1997, Virology 239, 389-401), Sindbis virus (Bredenbeek et al., 1993, J. of Virol. 67, 6439-6446), and Semliki Forest virus (Liljestrom and Garoff, 1991, Biotechnology 9, 1356-1361).

An αRP vector comprises a heterologous nucleic acid molecule encoding an antigen of interest, inserted into the viral genome. Transcription and translation of the nucleic acid molecule encoding the antigen that is comprised in αRP vector results in the antigen being expressed in cells infected with the αRP vector without producing a progeny, and in this way delivers and expresses heterologous antigen(s) to the immune system of the infected animal. The nucleic acid molecule may comprise an open reading frame (ORF) or a full gene encoding a complete protein, or may be fragment thereof encoding a section of a protein. The nucleic acid molecule encoding the antigen can be transcribed and expressed from an alphavirus subgenomic promoter, such as a 26S-alphavirus subgenomic promoter. Transcribed replicon RNA can be packaged into RPs by expression of the structural proteins by a packaging cell lines, or via co-transfection into suitable host cells of the replicon RNA and of one or more ‘helper’ RNA's encoding the structural proteins.

The αRP vector may comprise two or more heterologous nucleic acid molecule encoding the same or distinct antigens of interest inserted into its genome. This can be achieved in several ways. For example, such an αRP vector can encode a polycistronic reading frame, or can encode separate genes, e.g. by using one or more additional copies of the subgenomic promoter to allow expression of separate further protein(s).

αRP vectors may already be available, such as the commercially available VEEV based αRP vectors mentioned below, or the vector can be generated using well known techniques by incorporating the heterologous nucleic acid molecule encoding an antigen into a viral replicon backbone.

As used herein an antigen of an IAV-S is a protein or a fragment thereof that is able to trigger an immune response in a pig that protects the animal against a pathogenic infection with an IAV-S. The IAV genome consists of eight segments, which encode for at least 12 proteins. Three of these proteins are incorporated into the envelope of the virus: the viral hemagglutinin (HA), neuraminidase (NA), and matrix 2 (M2) proteins. HA and/or NA based vaccines are able to afford protective immunity to a pig. The HA protein is responsible for binding influenza virions to host cells. The function of the IAV-S NA protein is to cleave sialic acids on host cells, allowing newly made virions to be released efficiently from the infected cell. The transmembrane matrix 2 (M2) protein is a proton-selective ion channel, and is required for efficient uncoating of influenza A viruses (see review by Sandbulte et al., Vaccines 2015, 3, 22-73; doi: 10.3390 and Aguilar-Yáñez et al., PLOS ONE 2010; doi.org/10.1371/journal.pone.0011694).

Amino acid- and nucleotide sequences of IAV-S protein antigens can readily be obtained from publicly available literature and -sequences databases, such as from the NCBI (flu database), Influenza research database (IRD) or GenBank (GB).

The IAV-S antigen to be used in the present invention can be of any IAV-S(sub) type, -phylogenetic clade, cluster, -lineage, -strain or the like.

The term lineage as used herein refers to a set of influenza virus hemagglutinins or neuraminidase that have been grouped together (on the same branch) in an evolutionary tree that is rooted back to a similar (homologous) ancestor. These groupings have been made for European hemagglutinins and neuraminidase and are analogous to the phylogenetic clusters for U.S. viruses, but are not equivalent. Lineage determinations can be obtained by phylogenetic analysis of HA or NA sequences in question with pre-established reference sequences using readily available software, i.e., Clustal Omega (Sievers et al., 2011, Mol. Syst. Biol. 7, 539) or a web-accessible annotation tool for H1 HA sequences (Anderson et al., mSphere, 2016, 1 (6): e00275-16). In Europe there are three major lineages of H1 (Eurasian-avian like H1, Scotland/410440/1994-like H1 and pandemic 2009 like H1), one major lineage of H3 (Gent/1/1984-like H3), two major lineages of N1 (Eurasian Avian-like N1, Pandemic 2009 like N1), two major lineages of N2 (Gent/1/1984-like N2, Scotland/410440/1994-like N2) and two minor lineages of N2 (Italy/4675/2003 like N2, Human seasonal like N2). IAV-S classification rules- and tools for assessing classification are described in Watson et al., (J. Virol., 89, 9920-9931, 2015, doi: 10.1128/JVI.00840-15), https://journals.asm.org/doi/10.1128/mSphere.00275-16, and https://www.fludb.org/brc/h1CladeClassifier.spg?method=ShowCleanInputPage&decorator=influenza.

A pharmaceutically acceptable carrier is a biocompatible medium, viz. a medium that after administration does not induce significant adverse reactions in the treated subject, capable of presenting the antigen to the immune system of the animal after administration of the composition comprising the carrier. Such a pharmaceutically acceptable carrier may for example be a liquid containing water and/or any other biocompatible solvent or a solid carrier such as commonly used to obtain freeze-dried vaccines (based on sugars and/or proteins), optionally comprising an adjuvant.

An adjuvant is a compound or composition that is capable of providing a nonspecific stimulation to the immune system of an animal. Adjuvants are commonly used in vaccines based on inactivated- or subunit antigens. A wide variety of adjuvant types and compositions exist, for example: aluminium salts such as aluminium-hydroxide, or aluminium-phosphate, liposomes, glucans, alginate, bacterial components such as cell-wall components, oil-and-water emulsions of mineral- or non-mineral oils, synthetic adjuvants such as: non-ionic block polymers, polyamines such as dextran sulphate, Carbopol™, pyran, and Saponins, such as: Quil A™, or Q-vac™. Saponin and vaccine components may be combined in an ISCOM™. Furthermore, peptides such as muramyldipeptides, dimethylglycine, tuftsin, are often used as adjuvant. Similarly, combination products such as the ISA™ compositions (Seppic, France). More detailed information on adjuvants, their uses and effects can be found in the handbook: Vaccine adjuvants (Methods in molecular medicine, vol. 42, D. O'Hagan ed., 2000, Humana press, NJ, ISBN: 0896037355). For a detailed review on adjuvants used in pigs see: Charerntantanakul W., Vaccine, 2020; 38 (43), 6659-81).

### FURTHER EMBODIMENTS OF THE INVENTION

In an embodiment of the vaccine for use according to the present invention, the αRP is a Venezuelan Equine Encephalitis (VEE) αRP. In a more specific embodiment the αRP a TC-83 VEE αRP. The generation of VEE TC-83 αRP is for example described in U.S. Pat. Nos. 9,441,247 and 8,460,913. VEE based αRP vaccines are also the basis of several USDA-licensed vaccines, which include: Porcine Epidemic Diarrhea Vaccine, RNA (Product Code 19U5.P1), Swine Influenza Vaccine, RNA (Product Code 19A5.D0), Avian Influenza Vaccine, RNA (Product Code 19O5.D0), and Prescription Product, RNA Particle (Product Code 9PP0.00).

In a preferred embodiment of the vaccine for use as described above, the female pig is a sow or a gilt.

In again another embodiment the female pig is vaccinated twice. Vaccinating a female animal twice may increase the levels of antibodies that ultimately arrive in the piglets through uptake of colostrum, and such strategy does not pose serious problems in the everyday practice of keeping adult animals. In such a two-shot regime, the first (prime) vaccination is typically administered 5-8 weeks before expected parturition and boosted by a second vaccination 3-4 weeks later.

In still a further preferred embodiment the female pig is immunized while being pregnant.

Although it is foreseen that a female pig could receive adequate vaccination in between pregnancies in order to keep the level of antibodies (continuously) at an adequate height, it has shown to be useful to vaccinate the animal (at least once) while being pregnant. In particular, it has shown to be useful to vaccinate the pregnant female pig in a period of 5-8 weeks before parturition followed by a booster vaccination a, in particular in a period of 2-6 weeks before expected parturition, in both cases typically receiving the vaccine at least 1 or 3 weeks before expected parturition.

For female pigs that were already vaccinated during a previous pregnancy, a single revaccination can be carried out 1 to 4 weeks before expected parturition.

In another preferred embodiment the antigen of an IAV-S encoded by an αRP vector in a vaccine for use as described above is an HA antigen. The Examples herein demonstrate that HA antigens of IAV-S can advantageously be used in the vaccine to trigger a strong haemagglutinin inhibition (HI) antibody response in a female pig through active vaccination resulting in high levels of these antibodies in sera- and colostrum of pregnant female pigs. These HI antibodies are passively transferred to their progeny piglets reaching high levels of (systemic) HI antibody titers in piglet sera. Importantly, the maternally acquired antibodies are also capable of inducing local protection in the lungs of the piglets, as evidenced by a reduction of both virus load in the lungs and the lung lesions (lactogenic immunity).

In the context of the present invention an HA antigen of any IAV-S can be used, such as those disclosed in the art, for example, in publicly available sequence databases providing large numbers of amino acid sequences of IAV-S HA antigens and nucleotide sequences encoding such HA antigens (e.g. NCBI (flu database), Influenza research database (IRD) or GenBank (GB)).

An HA antigen of any of the four major lineages of IAV-S HA are of particular interest. These lineages are referred to as Scot/94 H1N2, EurAsianAvian H1N1, Gent1984 H3N2 and Pandemic2009 H1N1 (Watson et al., J. Virol., 89, 9920-9931, 2015, doi: 10.1128/JVI.00840-15). A vaccine for use as described above may comprise an HA antigen from any of these lineages.

An HA antigen of the Scot/94 lineage may be of any strain, such as from strain A/swine/Italy/3033-1/2015 (H1N2) or A/swine/France/35-140041 (H1N2). In a preferred embodiment the HA antigen of the Scot/94 lineage is from strain A/swine/Italy/3033-1/2015 (H1N2).

In another preferred embodiment the HA antigen comprises an amino acid sequence having at least 90%, preferably at least 93%, more preferably at least 95%, 96%, 97%, 98%, or 99% sequence identity with the HA antigen of Scot/94 reference strain A/swine/Italy/3033-1/2015 (H1N2): GB #ALX30160.1.

An HA antigen of the EA lineage may be of any strain, such as from strain A/swine/Denmark/101048-2/2011 (H1N1), A/swine/Italy/28762-3/2013 (H1N1) or A/swine/France/44-120070/2012 (H1N1). In a preferred embodiment the HA antigen of the EA lineage is from strain A/swine/Italy/28762-3/2013 (H1N1).

In another preferred embodiment the HA antigen comprises an amino acid sequence having at least 90%, preferably at least 93%, more preferably at least 95%, 96%, 97%, 98%, or 99% sequence identity with the HA antigen of EA reference strain A/swine/Italy/28762-3/2013 (H1N1): GB #AKJ81667.1.

An HA antigen of the Gent/84 lineage may be of any strain, strain A/swine/Italy/240849/2015 (H3N2) being preferred.

In another preferred embodiment the HA antigen comprises an amino acid sequence having at least 90%, preferably at least 93%, more preferably at least 95%, 96%, 97%, 98%, or 99% sequence identity with the HA antigen of Gent/84 reference strain A/swine/Italy/240849/2015 (H3N2): GB #ALX30415.1.

An HA antigen of the pdm09 lineage may be of any strain, strain A/swine/England/373/2010 (H1N1) being preferred.

In another preferred embodiment the HA antigen comprises an amino acid sequence having at least 90%, preferably at least 93%, more preferably at least 95%, 96%, 97%, 98%, or 99% sequence identity with the HA antigen of pdm09 reference strain A/swine/England/373/2010 (H1N1): GB #AFR76205.1.

In a particularly preferred embodiment the HA antigen of IAV-S to be used herein is any of the HA antigens described in Table 1 (below).

In an embodiment the present invention also provides a vaccine for use as described above, characterized in that the vaccine comprises at least one αRP vector encoding two or more distinct IAV-S antigens, in particular distinct HA antigens, more preferably HA antigens from different lineages, even more preferred the distinct HA antigens are of IAV-S strains belonging to lineages selected from the group Scot/94 H1N2, EurAsianAvian H1N1, Gent1984 H3N2 and pandemic2009 H1N1.

In a more specific form of this embodiment the vaccine comprises a first αRP vector encoding HA proteins of IAV-S strains belonging to two lineages selected from the group Scot/94 H1N2, EurAsianAvian H1N1, Gent1984 H3N2 and pandemic2009 H1N1 and a second αRP vector encoding HA proteins of IAV-S strains belonging to two other lineages of that group. In particular, the vaccine comprises a first αRP vector encoding HA proteins of IAV-S strains belonging to lineages Scot/94 H1N2 and EurAsianAvian H1N1 and a second αRP vector encoding HA proteins of IAV-S strains belonging to lineages Gent1984 H3N2 and pandemic2009 H1N1.

In still a further advantageous embodiment of the vaccine for use according to the invention as described above, the pharmaceutically acceptable carrier comprises an oil-and-water emulsion adjuvant. The Examples demonstrate that the presence of an oil-and-water emulsion adjuvant as a pharmaceutical acceptable carrier of the antigen in the vaccine has a positive effect on the generation of HI antibody titers in both the female pig and her piglet progeny.

The emulsion can be a water-in-oil (W/O) emulsion, where the oil is the continuous outer phase. Preferably, the emulsion is an oil-in-water (O/W) emulsion, where the oil is the dispersed internal phase. By the selection of the appropriate kind and concentration of emulsifier(s), such emulsions can be formed and stably maintained using standard emulsification techniques (see “Remington: the science and practice of pharmacy”, 2000, Lippincot, USA, ISBN: 683306472), and: Veterinary Vaccinology, Pastoret et al. ed., 1997, Elsevier, Amsterdam, ISBN 0444819681).

The oil-and-water emulsion adjuvant, when in the form of a preferred O/W emulsion facilitates the admixing of the emulsion with the αRP vector. For example, by the admixing of an aqueous- or freeze-dried composition comprising the αRP vector with the O/W emulsion. Simple handshaking for about 1 minute then suffices to properly mix the two aqueous compositions.

In a more specific embodiment the oil-and-water emulsion adjuvant may comprise a mineral oil or a non-mineral, biodegradable oil. Preferably, the mineral oil is a liquid paraffin oil (e.g. CAS number: 8042-47-5, generally available as Drakeol® 6VR (Penreco), Marcol® 52 (Exxon Mobile), and Klearol® (Sonneborn). Preferably, the non-mineral, biodegradable oil is selected from the group consisting of squalane, squalene, vitamin E, vitamin E-acetate, oleate, and ethyl-oleate, vitamin E-acetate being most preferred.

In still a further embodiment the oil-and-water emulsion adjuvant comprises two or more of a mineral oil and/or non-mineral, biodegradable oil, preferably the oil-and-water emulsion adjuvant comprises a liquid paraffin oil and vitamin E-acetate.

Examples of adjuvants useful in the vaccine for use as described above are the following proprietary O/W adjuvants: (micro) Diluvac Forte™ (based on a W/O emulsion of dl-α-tocopheryl acetate), XSolve™ (a combination of two O/W emulsion adjuvant components: Diluvac Forte which is based on vitamin E acetate (see EP 382.271) and Microsol™ which is based on liquid paraffin oil (see WO 2009/144.088), SVEA™ (W/O emulsion of squalane and Vitamin E-acetate, WO 2018/115.435) and ImpranFLEX™ (a water-in-oil adjuvant).

The vaccine for use according to the present invention comprises an immunologically effective amount of the oil-emulsion adjuvant that enhances the immune response triggered by the antigen of the vaccine. In an embodiment the vaccine comprises the oil-emulsion adjuvant in an amount of between about 10%-90% v/v of the vaccine. More preferably the vaccine comprises the oil adjuvant in an amount of between about 20%-80% v/v, 30-70% v/v, or even 40-60% v/v of the vaccine. Most preferred, the vaccine comprises the oil adjuvant in an amount of about 50% v/v of the vaccine.

The vaccine for use as described above comprises an immunologically effective amount of the αRP vector, such that the piglet progeny are protected against a pathogenic infection with IAV-S. In an embodiment the vaccine comprises from about 1×10\hat{ }3 to about 1×10\hat{ }11 RPs. In more particular embodiments, the vaccine comprises from about 1×10\hat{ }4 to about 1×10\hat{ }10 RPs. In even more particular embodiments, the vaccine comprises from about 1×10\hat{ }5 to about 1×10\hat{ }9 RPs.

In another embodiment, the vaccine for use as described above is administered in a 0.05 ml to 3 ml dose. In more particular embodiments, the dose administered is 0.1 ml to 2 ml. In still more particular embodiments, the dose administered is 0.2 to 2 ml.

The present invention also provides a vaccine for use as described above against multiple porcine pathogens. For example, the vaccine may comprise additional, inactivated-, attenuated or subunit antigens of other than IAV-S viral- and/or bacterial pig pathogens. Examples of such pig pathogens include porcine reproductive and respiratory syndrome virus (PRRS), porcine circovirus (PCV), transmissible gastroenteritis virus (TGE), porcine pseudorabies virus (PPRV), porcine parvovirus (PPV), porcine rotavirus (PRV), porcine epidemic diarrhea virus (PED), Pasteurella multocida of multiple serotypes, Salmonella spp., Escherichia coli, e.g., (serotypes K99, K88, 987P, or F41), Haemophilus parasuis, Lawsonia intracellularis, Mycoplasma spp. (e.g., Mycoplasma hyopneumoniae), Bordetella bronchiseptica, Erysipelas spp., Campylobacter spp., Actinobacillus pleuropneumoniae, Clostridium perfringens, and Clostridium difficile.

In a particularly preferred embodiment the vaccine additionally comprises antigens of E. coli and Clostridium perfringens.

In a further embodiment of the vaccine for use according to the present invention the vaccine is administered by a route customary for vaccination of pigs. In particular, by means of a parenteral administration. This includes subcutaneous-, intramuscular- and intradermal injections, intramuscular injections being preferred.

In another aspect the present invention provides a use of an alphavirus RNA replicon particle (αRP) vector encoding an antigen of an IAV-S for the manufacture of a vaccine to protect a piglet progeny of a female pig against a pathogenic infection with IAV-S by passive immunization of the piglet through active immunization of the female pig. All (combination of) features of this aspect are as outlined above.

In yet a further aspect the present invention provides method for passively immunizing a piglet progeny of a female pig against a pathogenic infection with IAV-S by actively immunizing the female pig with a vaccine comprising an alphavirus RNA replicon particle (αRP) vector encoding an antigen of an IAV-S. All (combination of) features of this aspect are as outlined above

The invention will now be further illustrated using the following specific examples.

### EXAMPLES

### Example 1 Preparation of an HA αRP Vector Based IAV-S Vaccine

### 1. Generation of Multi HA Genes αRP Vector.

The VEE replicon vectors used to express the HA and NA genes were constructed as previously described (U.S. Pat. Nos. 9,441,247, 8,460,913, WO 2019/121513 and WO2019110481) with the following modifications. The TC-83-derived replicon vector “pVEK” was digested with restriction enzymes Ascl and Pacl. For the dual-gene HA constructs, the selected open reading frame sequences were codon-optimized and synthesized with flanking Ascl and Pacl sites. Furthermore, the interstitial sequence between the two synthetic HA open-reading frames consisted of 47 nucleotides of non-coding heterologous sequence, and a second copy of the native TC-83 subgenomic (sg) RNA promoter and 5′ untranslated sgRNA region sequence. These dual-gene constructs were termed “pVDG” to differentiate them from the parental vector with a single sgRNA promoter sequence.

The following replicon particles were constructed:

TABLE 1
RNA particles, HA source strains, lineages, clade and GenBank #)

| RP | RP donor | | | |
|---|---|---|---|---|
| code | strain | Lineage | Clade | Accession # |
| EUH1-1 | A/swine/ | EurAsianAvian(EA) | 1C.2- | AKC43997.1 |
| | Denmark/ | | like | |
| | 10802-1/ | | | |
| | 2012(H1N2) | | | |
| EUH1-2 | A/swine/ | EurAsianAvian(EA) | 1C.2 | AKC43996.1 |
| | Denmark/ | | | |
| | 10-1048-2/ | | | |
| | 2011(H1N1) | | | |
| EUH1-3 | A/swine/ | Eurasian Avian(EA) | 1C.2.1 | AKJ81667.1 |
| | Italy/ | | | |
| | 28762-3/ | | | |
| | 2013(H1N1) | | | |
| EUH1-4 | A/swine/ | EurAsianAvian(EA) | 1C.2.1 | AKJ82257.1 |
| | France/ | | | |
| | 29-120326/ | | | |
| | 2012(H1N1) | | | |
| EUH1-5 | A/swine/ | EurAsianAvian(EA) | 1C.2.1 | AIL24876.1 |
| | France/ | | | |
| | 44-120070/ | | | |
| | 2012(H1N1) | | | |
| EUH1- | A/swine/ | EurAsianAvian(EA) | 1C.2.1 | AJW32121.1 |
| 13 | France/ | | | |
| | 56-140048- | | | |
| | 2014(H1N1) | | | |
| EUH1-9 | A/swine/ | Pandemic 2009 | 1A.3.3.2 | AGG86840.1 |
| | Moeglingen/ | (pdm09) | | |
| | IDT14859/ | | | |
| | 2012(H1N2) | | | |
| EUH1- | A/swine/ | Pandemic 2009 | 1A.3.3.2 | AFR76205.1 |
| 11 | England/373/ | (pdm09) | | |
| | 2010(H1N1) | | | |
| EUH1- | A/swine/ | Pandemic 2009 | 1A.3.3.2 | ADA70669.1 |
| 12 | Italy/ | (pdm09) | | |
| | 290271/ | | | |
| | 2009(H1N1) | | | |
| EUH1-8 | A/swine/ | Scotland 1994- | 1B.1.1 | AKJ81533.1 |
| | England/ | clade 1 (Scot94-1) | | |
| | 9953/ | | | |
| | 2012(H1N2) | | | |
| EUH1-6 | A/swine/ | Scotland 1994- | 1B.1.2.2 | AGR45140.1 |
| | Italy/ | clade 2 (Scot94- | | |
| | 186822/ | 2.2) | | |
| | 2011(H1N2) | | | |
| EUH1- | A/swine/ | Scotland 1994- | 1B.1.2.2 | ALX30160.1 |
| 17 | Italy/ | clade 2 (Scot94- | | |
| | 3033-1/ | 2.2) | | |
| | 2015(H1N2) | | | |
| EUH1-7 | A/swine/ | Scotland 1994- | 1B.1.2.3 | AHI43247.1 |
| | France/ | clade 3 (Scot94- | | |
| | 22-130212/ | 2.3) | | |
| | 2013(H1N2) | | | |
| EUH1- | A/swine/ | Scotland 1994- | 1B.1.2.3 | AIL24895.1 |
| 15 | France/ | clade 3 (Scot94- | | |
| | 35-140041/ | 2.3) | | |
| | 2014(H1N2) | | | |
| EUH3-1 | A/swine/ | Gent 1984 | 3.1970.1 | AKJ83041.1 |
| | Netherlands/ | (Gent/84) | | |
| | Ysselsteyn- | | | |
| | CVI8864A/ | | | |
| | 2012(H3N2) | | | |
| EUH3-2 | A/swine/ | Gent 1984 | 3.1970.1 | AKJ82900.1 |
| | Belgium/ | (Gent/84) | | |
| | Glabbeek- | | | |
| | 284/ | | | |
| | 2012(H3N2) | | | |
| EUH3-3 | A/swine/ | Gent 1984 | 3.1970.1 | AKJ83006.1 |
| | Spain/ | (Gent/84) | | |
| | 33936/ | | | |
| | 2012(H3N2) | | | |
| EUH3-4 | A/swine/ | Gent 1984 | 3.1970.1 | ALX30415.1 |
| | Italy/ | (Gent/84) | | |
| | 240849/ | | | |
| | 2015(H3N2) | | | |
| EUSIV- | A/swine/ | Gent1984/ | 3.1970.1/ | ALX30415.1/ |
| K | Italy/ | Pandemic 2009 | 1A.3.3.2 | AFR76205.1 |
| | 240849/ | | | |
| | 2015(H3N2)/ | | | |
| | A/swine/ | | | |
| | England/373/ | | | |
| | 2010(H1N1) | | | |
| EUSIV- | A/swine/ | Scot94-2.3/EA | 1B.1.2.2/ | ALX30160.1/ |
| T8 | Italy/3033-1/ | | 1C.2.1 | AKJ81667.1 |
| | 2015(H1N2)/ | | | |
| | A/swine/ | | | |
| | Italy/ | | | |
| | 28762-3/ | | | |
| | 2013(H1N1) | | | |

αRP vectors EUSIV-K and RP EUSIV-T8 were used to determine the immunogenicity and efficacy of a multivalent IAV-S vaccine comprising the two-dual-HA RPs.

### 2. Preparation of the αRP Vector Based Vaccine.

RP particles dissolved in PBS 0.01M with Phenol Red of batch number RP EUSIV-K and RP EUSIV-T8 were mixed 1:1 (v/v) with X-Solve® adjuvant (O/W emulsion of 21% v/v of the non-metablizable oil Marcol® 52 and 1,25% v/v of the metabolizable oil vitamin-E acetate, available from MSD Animal Health, Boxmeer, The Netherlands) or Micro Diluvac Forte® adjuvant (O/W emulsion of 7.5% v/v of the metabolizable oil vitamin-E acetate) in Porcilis® ColiClos vaccine (available from MSD Animal Health, Boxmeer, The Netherlands). Porcilis® ColiClos vaccine comprises multiple E. coli antigens (F4ab/F4ac/F5/F6 fimbrial adhesins) and an LT toxoid antigen of C. perfringens.

### Example 2 Lactogenic/Passive Immune Protection of Piglets Against Influenza Virus

This experiment evaluated the lactogenic immune protection of piglets against influenza A virus induced by multi-gene IAV-S HA RNA particles mixed with 50% (v/v) Xsolve adjuvant or Porcilis® ColiClos vaccine (micro Diluvac Forte® adjuvant).

### Study Design.

Twelve high health pregnant gilts (not previously vaccinated against Clostridium perfringens type C and/or E. coli and have no/low antibody levels against IAV-S), which were expected to give birth to 8 to 10 piglets, were purchased from a suitable commercial farm. The gilts were vaccinated (5*10⁶ RP/dose per RP) at 6 and 2 weeks pre-farrow either intradermally (i.d.) or intramuscularly (i.m.): Table 1. A blood sample from all gilts was collected at 6 and 2 weeks pre farrowing (WPF) and on the day of farrowing to determine antigen specific HI titers. In addition, colostrum samples were collected on the day of farrow and a milk sample was collected at 3 days post farrow.

Piglets: Blood samples from all piglets was collected at approximately one week of age (WOA) (90) and on the day of challenge (88) to determine antigenic specific HI titers. Once the piglets reach about 4 WOA, they were inoculated intratracheally (i.t.) with IAV-S challenge strain A/swine/France/53-130065/2013 (H1N1), (10⁵ TCID50 per pig in 5 mL PBS). A maximum of 10 piglets per litter were challenged. Nasal swabs were collected from pigs at the day of challenge (i.e., 0) and 1, 2 and 3 days post challenge to determine virus shedding.

One part of the piglets (24) were necropsied at 24 hours post challenge and the other part (61) at 72 hours post challenge. At necropsy, lung lesions were scored and six lung samples from each pig (one each from the left cranial, middle and caudal lobe, and one each from the 3 corresponding right lung lobes) were collected to measure the viral load in the lungs.

TABLE 2
Experimental groups

| | | No. of | Vaccine | | | |
|---|---|---|---|---|---|---|
| | No. | piglets at | (6 and 2 | O/W | | |
| | of | farrow | weeks | emulsion | | |
| Group | gilts | date | pre-farrow) | Adjuvant | Application | |
| 1 | 3 | 22 | IAV-S RP + | μDF | 2 | mL/i.m. |
| | | | ColiClos | | | |
| 2 | 3 | 33 | IAV-S RP | XSolve50 | 2 | mL/i.m. |
| 3 | 3 | 20 | IAV-S RP | XSolve50 | 200 | μl/i.d. |
| 4 | 3 | 22 | PBS + | μDF | 2 | mL/i.m |
| | | | ColiClos | | | |

### Experimental Procedures

### 1. IAV-S HI Antibody Titers in Sera and Colostrum.

The two dual HA gene RPs were tested for their in vivo functionality by monitoring the HI titers in the sera of the vaccinated pigs. Representative IAV-S strains from four IAV-S lineages with >85% amino acid identity to HA-RPs were used in the HI test. Next to the lineage and the strain name, the clade classification and percentage amino acid identity of the IAV-S strain used for the quantification of HI titers to the respective vaccine component is mentioned in table 2. IAV-S specific antibodies in pig serum samples were determined by HI tests by standard procedures. In short, sera were pretreated with periodate to remove non-specific inhibitors. Subsequently, pretreated sera were two-fold serially diluted and incubated with influenza virus strain (Table 2). After incubation step, chicken erythrocytes were added, incubated and plates were read for inhibition of agglutination. The reciprocal of the highest serum dilution that completely inhibited erythrocyte agglutination was assigned as the HI titer and expressed in log base 2 values.

TABLE 3
Heterologous IAV-S strains used in HI test

| | | Strains used | Clade | % |
|---|---|---|---|---|
| RP code | HA sequence strain | for HI assay | (fludb) | identity |
| EUH1-3 | A/swine/Italy/ | A/swine/France/ | 1C.2.1 | 97.0 |
| | 28762-3/2013 | 53-130065/2013 | | |
| | (H1N1) | (H1N1) | | |
| EUH1-17 | A/swine/Italy/ | A/swine/France/ | 1B.1.2.3 | 91.7 |
| | 3033-1/2015 | 22-130212/2013 | | |
| | (H1N2) | (H1N2) | | |
| EUH1-11 | A/swine/England/ | A/swine/Mn/ | 1A.3.3.2 | 97.5 |
| | 373/2010 (H1N1) | A01483170/2014 | | |
| | | (H1N1pdm) | | |
| EUH3-4 | A/swine/Italy/ | A/swine/Belgium/ | 3.1970.1 | 97.5 |
| | 240849/2015 | 113/2013 (H3N2) | | |
| | (H3N2) | | | |

### 2. Antibody Titers Against Escheria Coli in Sera and Colostrum.

Antibody titers in the sera against E. coli antigens in the vaccine (types K88ab, K88ac, K99, 987P and LT) were determined by ELISA according to standard procedures.

3. Antibody Titers Against Clostridium perfringens in Sera and Colostrum.

Antibody titers in the sera against Clostridium perfringens type C beta toxin was determined by ELISA according to standard procedures.

### 4. Virus Quantification in Clinical Samples.

Nasal swabs and lung tissues were tested for infectious titer by serial dilution of the original nasal swab and 20% lung tissue homogenate samples. Two lung homogenates (one prepared by pooling equivalent quantity of samples collected from the left cranial, middle and caudal lobes and the other prepared from the corresponding right lung lobes) per animal were tested. In short, samples were 10-fold serially diluted with IAV-S infecting media. Each dilution was inoculated onto three replicate wells of confluent MDCK monolayers. Plates were incubated at 37° C. with 5% CO₂ for 4 days and the presence of virus in the supernatant was detected by hemagglutination. IAV-S titers were calculated as log₁₀ TCID₅₀ per mL, using the Spearman-Kärber method.

### 5. Macroscopic Lung Scoring.

Macroscopic lung lesions indicative of Swine Influenza were scored based on the percentage of abnormal lung tissue per lung lobe and a weighted score was allocated to each of the seven lobes of lungs according to the relative weight of the lung lobes.

### Results.

### 1. IAV-S Haemagglutination Inhibition Titers Gilts in Serum, Colostrum and Milk.

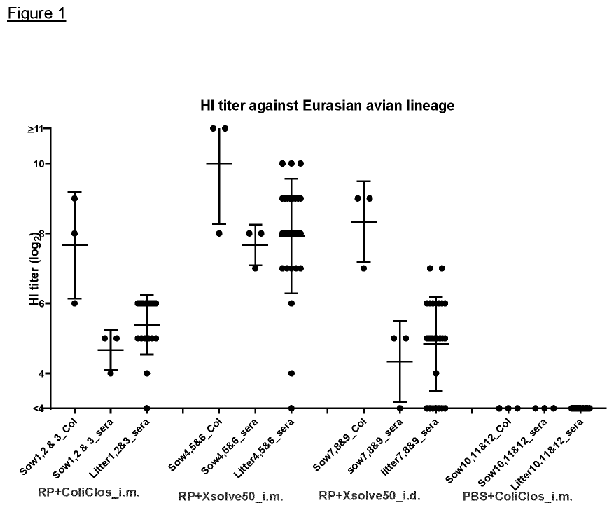

A blood sample from all gilts was collected at 6 and 2 weeks pre farrowing (WPF) and on the day of farrowing (FD) to determine antigen specific HI titers. Colostrum samples were collected on the day of farrow and a milk sample was collected at 3 days post farrow. The tested IAV-S vaccine (based on EUSIV-T8 and EUSIV-K) induced HI titers against all representative heterologous strains from respective lineages in serum, colostrum and milk. (IAV-S HI titers gilt sera of 8 log₂ EurAsian, 9 log₂ Gent84, 11 log 2 pandemic, 5 log 2 Scot94 Clu1 and 7 log₂ against Scot94 Clu2). Measured average HI titers and standard deviation per group for the EU IAV-S strain of the gilts are given in FIG. 1.

In general, IAV-S vaccine induced higher HI titers against all four IAV-S strains when mixed and applied with XSolve50 adjuvant than with Porcilis® ColiClos adjuvant.

Further, it was observed that administration of the IAV-S vaccine via the intramuscular route induced higher HI titers than administered via the intramuscular route.

### 2. IAV-S Haemagglutination Inhibition Titers Piglets in Serum.

Blood samples were collected from all piglets at approximately one week of age and on the day of challenge (approximately 4 WOA). HI titers against all representative heterologous strains from respective lineages in the offspring was measured at approximately one week of age as well as on the day of challenge. Similar to the IAV-S HI titers in the gilt sera, the offspring of the gilts vaccinated with IAV-S vaccine Xsolve50 adjuvant had the highest HI titers against all lineages among all three test groups followed by the group that received IAV-S vaccine mixed with Porcilis® Coliclos vaccine via i.m route and IAV-S vaccine with Xsolve50 adjuvant via i.d. route. Furthermore, the antibody titers in the sera collected at one week of age were higher than the sera collected at 4 WOA. Summarized IAV-S HI antibody titers (EA lineage) measured on 1 WOA are presented in FIG. 1.

3. Antibody Titers in Piglets Against E. coli and Clostridium perfringens Type C in Serum.

Piglet sera samples were tested for antibody titers against five E. coli five antigens and C. perfringens at approximately one week of age as well as on the day of challenge of group 1 and 4 (IAV-S vaccine in combination with Porcilis® ColiClos or with only the Porcilis® ColiClos vaccine respectively). Blood samples were collected from all piglets at approximately 1 WOA and 4 WOA (day of challenge).

The sera samples of piglets of the gilts that had received Porcilis® ColiClos vaccine alone or in combination with IAV-S vaccine had highest antibody titers against all E. coli types when compared to the other experimental groups. In general, no marked differences in the measured E. coli antibody titers were observed between the group that received Porcilis® ColiClos alone or in combination with IAV-S vaccine. Furthermore, the antibody titers in the sera collected at one week of age were higher than the sera collected at 4 WOA.

The sera samples of piglets of the gilts that had received Porcilis® ColiClos vaccine alone or in combination with IAV-S vaccine had highest antibody titers against all E. coli types when compared to the other experimental groups. In general, no marked differences in the measured C. perfringens antibody titers were observed between the group that received Porcilis® ColiClos alone or in combination with IAV-S vaccine. Furthermore, the antibody titers in the sera collected at one week of age were higher than the sera collected at 4 WOA.

4. Protection Against Lung Lesions in Piglets after IAV-S Challenge.

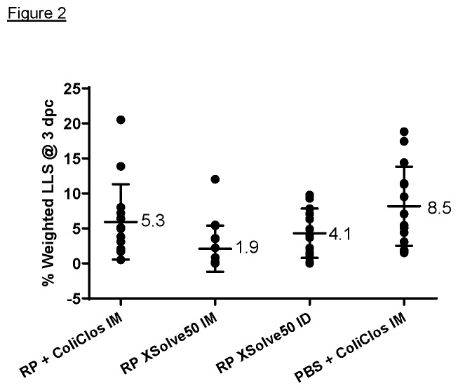

Four weeks old off-springs of the vaccinated gilts were intra-tracheally infected with IAV-S EA lineage strain A/swine/France/53-130065/2013 (H1N1) and were necropsied either at 1 or 3 days post infection to evaluate IAV-S infection induced lung lesions. The evaluated lung lesion scores at 3 post infection are summarized in FIG. 2.

The data revealed that all off-springs of gilts that had received IAV-S vaccine either alone or in combination with Porcilis® Coliclos vaccine had lower LLS compared to non-IAV-S vaccinated group. Off-springs of the group that had received IAV-S vaccine with XSolve50 adjuvant via i.m route (group 2) had the lowest lung lesions scores followed by the groups that had received either IAV-S vaccine with Xsolve50 adjuvant via i.d. route (group 3) or IAV-S vaccine with Porcilis® ColiClos (group 1).

5. Reduction of Viral Load in Lungs Piglets after IAV-S Challenge.

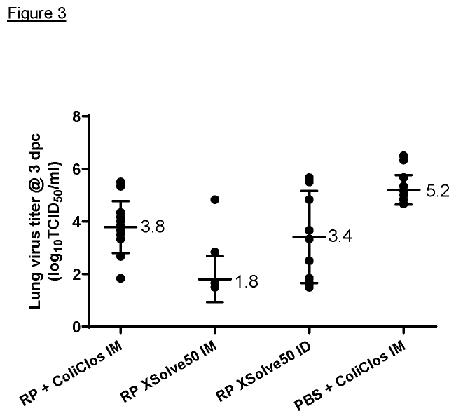

The measured titration data obtained from the lung homogenates at 3 post infection are summarized in FIG. 3. The data revealed that all off-springs of gilts that had received IAV-S vaccine either alone or in combination with Porcilis® Coliclos vaccine had lower levels of virus in lung homogenates compared to non-IAV-S vaccinated group. Off-springs of the group that had received IAV-S vaccine with XSolve50 adjuvant via i.m route (group 2) had the lowest viral load in the lung homogenates followed by the groups that had received either IAV-S vaccine with Xsolve50 adjuvant via i.d. route (group 3) or IAV-S vaccine with Porcilis® ColiClos (group 1).

### CONCLUSION

IAV-S RP (EUSIV-T8 and EUSIV-K) vaccine induced high sera and colostrum HI titers against all tested representative heterologous IAV-S strains from respective lineages. A direct correlation between HI titers in gilt sera, colostrum and piglet sera was observed.

IAV-S vaccine induced higher HI titers against all four IAV-S strains when mixed and applied with XSolve50 adjuvant instead of Porcilis® ColiClos.

The magnitude of antibody titers induced by Porcilis® ColiClos against all five Escheria coli types and Clostridium ß-toxin was not affected when it was applied in combination with IAV-S RP vaccine.

Intramuscular route of application of IAV-S vaccine induced higher HI titers (both in sera and colostrum) against all four strains when compared to intradermal route.

The HI titers measured in the sera of the piglets correlated with the reduction of lesion and viral load in the lungs.

### Example 3 Lactogenic/Passive Immune Protection of Piglets Against Porcine Epidemic Diarrhea Virus (PEDV)

This experiment evaluated the lactogenic/passive immune protection of piglets against PEDV induced by vaccination with RNA particles encoding PEDV spike glycoprotein gene in Xsolve50 adjuvant.

### Study Design.

Twenty-four PED serologically and clinically free sows were vaccinated intramuscularly at 8, 4 and 2 weeks to farrow with RNA particles encoding PEDV spike glycoprotein gene (lyophilized T9 RNA-based vaccine comprising an alphavirus RNA RP based on a replicon construct from VEEV strain TC-83. This was constructed to comprise the coding sequence of the spike protein of US virulent PEDV strain AH2012 (see GenBank Accession number KC210145). 1 mL per dose and mixed with X-Solve to be 50% at point of use). Pigs were challenged IT at 3-5 days of age with CO/2013 strain (10⁶ TCID50 per pig).

TABLE 4
Experimental groups

| | Target | Potency |
|---|---|---|
| Group | (Log 10 copy/mL) | (Log 10 copy/mL) |
| Vaccine | 6.3 | 6.1 |
| Placebo | — | — |

### Results

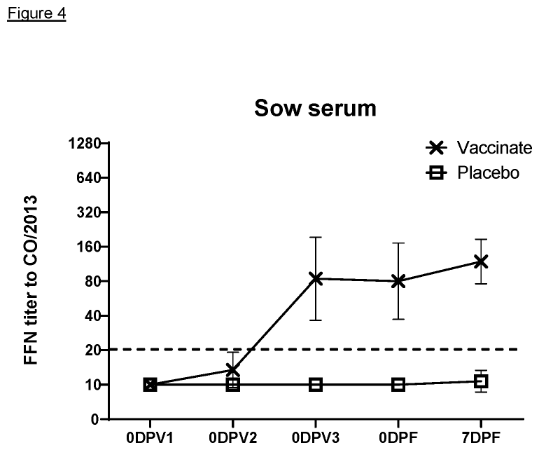

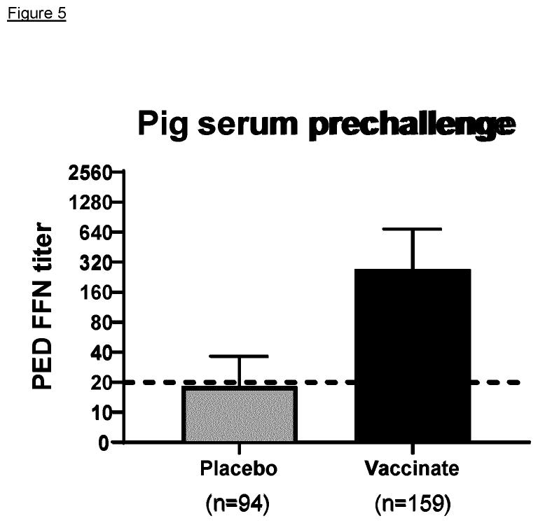

The vaccine induced a moderate level (between 80-160) of serum PED neutralization (PED SN) titer post 3 vaccinations (FIG. 4). FFN (fluorescent focus neutralization) assay is used to determine PEDV serum neutralization titer. Piglets born from vaccinated sows had PED-maternally derived antibodies (80-640) against the PED challenge strain at the timepoint before challenge (FIG. 5).

### CONCLUSION

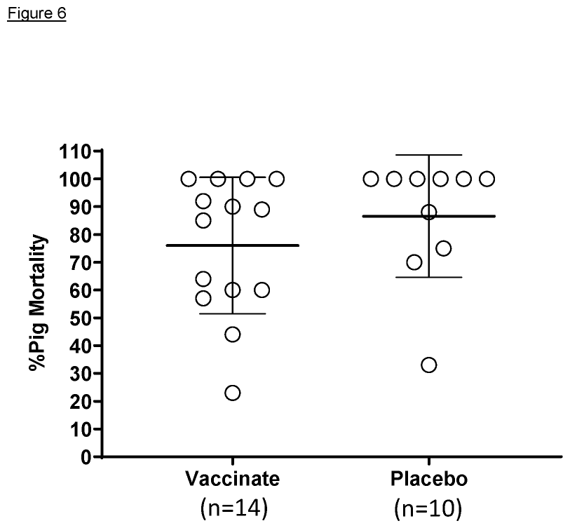

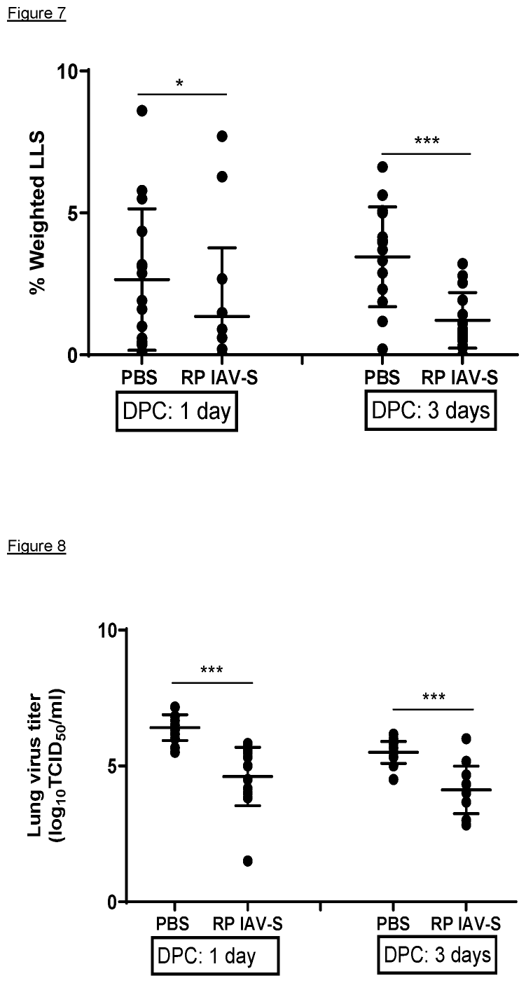

However, despite the generation in sows of—and transfer of SN antibodies to their piglets, the vaccine failed to protect suckling pigs against PED-induced mortality (Table 5 and FIG. 6).

TABLE 5
Mortality data

Pig mortality

| Groups | Mean | Median |
|---|---|---|
| Vaccinate | 76% | 87% |
| Placebo | 86.6% | 100% |

### Example 4 Lactogenic/Passive Immune Protection of Piglets Against Influenza Virus

Completely corresponding to example 2, this experiment evaluated the lactogenic immune protection of piglets against influenza A virus induced by multi-gene IAV-S HA RNA particles mixed with 50% (v/v) Xsolve adjuvant by vaccinating multiparity sows and demonstrating the passive protection, but against an alternative IAV-S challenge strain, viz. A/swine/Belgium/113/2013 (H3N2) at 6 WOA.

FIG. 7 shows the weighted lung lesion scores (LLS) with mean±SD of the piglets measured at 1 or 3 days post challenge with. Average values±SD were based on 15 animals belonging to six litters each. Six sows per group were either vaccinated at 6 and 2 weeks pre-farrow or served as non-vaccinated controls. Born off-springs were infected with A/swine/Belgium/113/2013 (H3N2) IAV-S at 6 WOA and necropsied at 1 or 3 days post infection to evaluate lung lesion scores.

FIG. 8 shows the IAV-S titers with mean±SD of the piglet lung tissues collected at 1 or 3 days post challenge. Average values±SD were based on15 animals per group belonging to six litters each. Six sows per group were either vaccinated at 6 and 2 weeks pre-farrow or served as non-vaccinated controls. Born off-springs were infected with A/swine/Belgium/113/2013 (H3N2) IAV-S at 6 WOA and necropsied at 1 or 3 days post infection to the measure viral load in the lungs.

It is clear that a good immune response and protection is induced against the challenge strain.

1. A method to protect a piglet against a pathogenic infection with swine influenza A virus (IAV-S) comprising passive immunization of the piglet through ingestion of colostrum or milk from a female pig actively immunized with a vaccine comprising an immunogen and a pharmaceutically acceptable carrier, wherein the immunogen is an alphavirus RNA replicon particle (aRP) vector encoding an antigen of an IAV-S.

2. The method according to claim 1, characterized in that the female pig is a sow or gilt.

3. The method according to claim 1, characterized in that the antigen is an IAV-S haemagglutinin (HA) protein.

4. The method according to claim 1, characterized in that the αRP vector encodes two or more distinct IAV-S antigens.

5. The method according to claim 1, characterized in that the vaccine comprises two or more distinct αRP vectors encoding distinct IAV-S antigens.

6. The method according to claim 1, characterized in that the antigen or antigens are of IAV-S strain(s) belonging to a lineage selected from the group consisting of Scot/94 H1N2, EurAsianAvian H1N1, Gent1984 H3N2 and pandemic2009 H1N1.

7. The method according to claim 6, characterized in that the vaccine comprises a first αRP vector encoding HA antigens of IAV-S strains of two of the lineages selected from the group consisting of Scot/94 H1N2, EurAsianAvian H1N1, Gent1984 H3N2 and pandemic2009 H1N1 and a second αRP vector encoding HA antigen of IAV-S strains of the two other lineages of that group.

8. The method according to claim 7, characterized in that the first αRP vector encode HA antigens of IAV-S strains of the lineages Scot/94 H1N2 and EurAsianAvian H1N1 and the second αRP vector encodes HA antigens of IAV-S strains of the lineages Gent1984 H3N2 and pandemic2009 H1N1.

9. The method according to claim 1, characterized in that the pharmaceutically acceptable carrier comprises an oil-and-water emulsion adjuvant.

10. The method according to claim 1, characterized in that the vaccine is administered intra-muscularly.

11. The method according to claim 1, characterized in that the vaccine additionally comprises an antigen of E. coli.

12. The method according to claim 1, characterized in that the vaccine additionally comprises an antigen of Clostridium perfringens.

13. A method of manufacturing an IAV-S vaccine comprising combining an alphavirus RNA replicon particle (αRP) vector encoding an antigen of an IAV-S with a pharmaceutically acceptable carrier.

14. A method for passively immunizing a piglet progeny of a female pig against a pathogenic infection with IAV-S by actively immunizing the female pig with a vaccine comprising an alphavirus RNA replicon particle (αRP) vector encoding an antigen of an IAV-S
